(19) United States
(12) Patent Application Publication
(10) Pub. No.: US 2003/0198616 A1
(43) Pub. Date: Oct. 23, 2003

(54) Moisturizing skin gel and method

(75) Inventor: John R. Howard, Ridgefield, CT (US)

Correspondence Address: FITZPATRICK CELLA HARPER & SCINTO, 30 ROCKEFELLER PLAZA, NEW YORK, NY, 10112, US

(73) Assignee: Combe Incorporated, White Plains, NY (US)

(21) Appl. No.: 10/127,588

(22) Filed: Apr. 23, 2002

(51) Int. Cl. A61K 31/4152; A61K 7/06; A61K 7/11

(52) U.S. Cl. 424/70.13; 424/70.16; 514/386

(57) ABSTRACT

A skin moisturizing composition comprising water, a skin moisturizer, a water soluble hydroxyalkylcellulose polymer and additives is free of fats and oils. The composition derives substantial water-barrier properties from the water soluble hydroxyalkylcellulose polymer.

### BACKGROUND OF THE INVENTION

1. Field of the Invention

The invention relates to the field of skin-care compositions and methods of preventing transepidermal water loss. The preferred embodiment utilizes an aqueous skin care composition in the form of a gel free from occlusive fats or oils which provides substantial moisture barrier properties.

2. Description of the Prior Art

Nonvolatile hydrocarbons such as petrolatum, mineral oil, paraffin wax, ozokerite and the like have long been used in skin creams and lotions. These materials function as emollients by covering the skin with a hydrophobic occlusive film which prevents water loss from the skin surface to the environment. Likewise, animal fats and oils such as lanolin and its various derivatives, such as acetylated lanolins, have also been used in skin creams and lotions as emollients, depositing films on the skin that are hydrophobic, waxy and protective. The drawback of the conventional occlusive-type moisture barriers containing fats and/or oils is that they generally impart to the skin an uncomfortable feeling of warmth in addition to a sticky, oily, greasy or waxy feel.

U.S. Pat. No. 4,837,019 discloses skin treatment compositions which are said to avoid the problem of greasy feel and to counteract moisture loss and promote healing of burned or sunburned skin. The compositions include a moisturizing component formed of polyglycerylmethacrylate, glycerine, allantoin, panthenol, amino acid complex, and fibronectin. The skin treatment composition disclosed in this patent also comprises non-functional components, and in one embodiment, the moisturizing component is included in an aqueous gel.

U.S. Pat. No. 4,963,591 discloses the addition of a water insoluble cellulosic polymer/solvent system to non-aqueous skin care formulations. An example of the polymer solvent system is Ethocel Standard™, a material marketed by Dow Chemical Company, having an ethoxyl content of from 48.0 to 49.5%, in amounts ranging from about 0.75% to about 1.60% by weight of the total composition, and a solvent for the cellulosic polymer such as ethanol, propanol or isopropanol, present in amounts ranging from about 20% up to about 95% by weight or more of the total composition. The polymer/solvent system disclosed in the aforesaid 4,963,591 patent, in combination with well-known cosmetic components, is said to permit the dispersion of a fine, thin, substantive film over the skin surface which is free of any sticky, oily, greasy or waxy feel. However, all of the disclosed compositions are non-aqueous.

### SUMMARY OF THE INVENTION

The skin moisturizing composition according to the invention is aqueous, has substantial water barrier properties, and comprises (in addition to water) a water soluble hydroxyalkylcellulose polymer and a skin moisturizer, such as glycerin. The composition does not require a film forming polyglycerylmethacrylate polymer, occlusive fats and/or oils.

In particularly preferred embodiments the composition is a gel containing between about 80 to about 90 percent by weight deionized water, between about 1 to about 3 percent by weight hydroxyethylcellulose or an equivalent, about 8 to about 12 percent by weight glycerine and about 0.1 to about 2 percent by weight emulsifier. Other ingredients, functional for other purposes besides moisturization, may be added in relatively minor amounts, including, without limitation, antihistamines, vulnerary agents, antipruritics, anesthetics, stabilizers, preservatives, antimicrobials, antibacterials, antiseptics, antioxidants and UV filters in a combined amount from about 1.0 percent by weight to about 8.0 percent by weight, preferably less than about 3.0 percent by weight.

The invention also includes a method of reducing transepidermal water loss without applying a film- forming methacrylate polymer, occlusive fats or oils. The method comprises topically administering to skin in need thereof a therapeutically effective amount of an aqueous composition comprising a water soluble hydroxyalkylcellulose polymer and glycerine to reduce transepidermal water loss without imparting an oily feel to the skin.

### BRIEF DESCRIPTION OF THE FIGURES

FIG. 1 depicts the percentage improvement in transepidermal water loss properties of a composition according to the invention versus a prior art composition having occlusive oils.

FIG. 1a depicts the percentage improvement in moisturization properties of a composition according to the invention versus a prior art composition having occlusive oils.

FIG. 2 depicts a comparison of moisturization properties of compositions according to the invention, and comparative examples without a vulnerary agent, and without a moisturizing agent.

FIG. 2a depicts a comparison of water barrier properties of compositions according to the invention, and comparative examples without a vulnerary agent, and without a moisturizing agent.

FIG. 3 depicts a comparison of the moisturization properties of a water soluble hydroxyalkylcellulose polymer versus carbomer gelling agent.

FIG. 3a depicts a comparison of the water barrier properties of a water soluble hydroxyalkylcellulose polymer versus carbomer gelling agent.

### DESCRIPTION OF THE PREFERRED EMBODIMENTS

The skin-care composition according to the invention is free of occlusive fats or oils and, consequently, does not have the greasy, oily feel of many prior art moisturizers. Broadly speaking, the fats or oils which are not present in the inventive composition are those which impart an oily, sticky or waxy feel to the skin.

For the purpose of this invention the inventive composition is deemed free from such fats or oils when it contains less than about 0.10 percent by weight of hydrocarbon oils and waxes, such as mineral oil, petrolatum, paraffin, ceresin, ozokerite and the like; vegetable and animal fats and oils such as castor oil, cocoa butter, safflower oil, cottonseed oil, corn oil, olive oil and the like; C₁₀-C₂₀ fatty acids; alkyl or alkenyl esters of C₁₀-C₂₀ fatty acids; C₁₀-C₂₀ fatty alcohols; lanolin and its derivatives such as lanolin oil, lanolin wax, lanolin alcohols, lanolin fatty acids, etc.; wax esters such as beeswax and the like; and vegetable waxes such as carnauba and candelilla waxes, sterols, phospholipids, fatty amides and the like.

More preferably, the composition contains undetectable amounts of such fats and/or oils.

Notwithstanding that these conventional occlusive materials are avoided, the inventive composition unexpectedly provides substantial moisture barrier properties.

The phrase “substantial moisture barrier properties,” as used herein is understood to mean that a topical dosage on the order of 2 mg/cm² results in an improvement in transepidermal water loss of greater than about 30% over a baseline measurement.

The phrase “transepidermal water loss” (TEWL) as employed herein refers to water loss through the epidermis generally exacerbated by skin barrier damage caused by environmental factors associated with dry skin, such as detergents, soaps, solvents or ultraviolet light which tend to remove or damage the skin's protective lipids, resulting in such water loss.

The phrase “substantial evaporative water loss”, as used herein means a loss of more than 2.5 g H₂O/m²/hr measured using a vapor pressure gradient estimation system as described herein.

As employed herein the phrase “skin moisturizer” refers to a compound which moisturizes the skin; that is, which tends to enhance the skin's water content.

While many components are used in various embodiments, as described below, the critical components of the skin care composition according to the invention are water, a water soluble hydroxyalkylcellulose polymer and a moisturizer, such as glycerin. The composition is solubilized in an aqueous carrier. Such an aqueous carrier is composed principally of water, preferably deionized water, and may contain other water-compatible solvents. Preferably, the composition is more than 50 percent by weight of water. Preferably greater than about 80 percent by weight of the composition is deionized water. Most preferably between 80 and 90 percent by weight of the composition is deionized water.

Preferably the aqueous skin moisturizing composition is employed in the form of a gel. Gel, as used herein, connotes a semi-solid form of the composition, more solid than a lotion, but readily smoothed over the skin.

The water soluble hydroxyalkylcellulose polymer used in the moisturizing composition of the invention typically performs a dual function of gelling the composition and forming a moisture barrier to reduce TEWL. Preferably, the water soluble hydroxyalkylcellulose polymer is a lower alkyl (C1-C8) hydroxyalkyl cellulose, such as hydroxymethylcellulose, hydroxyethylcellulose, or hydroxypropylcellulose. Preferred water soluble hydroxyalkylcellulose polymers at a concentration of 2 percent by weight in water exhibit a viscosity at room temperature between about 10,000 centipoise and about 250,000 centipoise. Hydroxyethylcellulose is preferred. A particularly preferred hydroxyethylcellulose is commercially available under the trade name NATROSOL 250 HNF or NATROSOL HX available from Hercules Chemical Company, New York, N.Y.

Generally the water soluble hydroxyalkylcellulose polymer is included in the composition in an amount between about 1.0 percent by weight and about 5.0 percent by weight of the composition. In a preferred embodiment, the composition comprises about 2.0 weight percent hydroxyethylcellulose.

The skin moisturizer (or humectant), such as glycerin, (or glycerine or glycerol, as it is sometimes referred to), is generally present in amounts between about 1.0 percent by weight up to about 20.0 percent by weight of the composition, preferably between about 8.0 and about 12.0 percent by weight. In a preferred embodiment, glycerine (96-100%) is present in the composition in an amount of about 10.0 percent by weight.

Other skin moisturizers, including polyhydric alcohols known to have a similar humectant effect to glycerin, such as alkylene polyols and their derivatives, including propylene glycol, sorbitol, ethoxylated glycerol and mixtures thereof, may be employed.

Conventional additives may be advantageously included in the composition including, without limitation, antihistamines, antimicrobials, antiseptics, antipruritics, anesthetics, emulsifiers, vulnerary agents, antioxidants and UV filters and stabilizers. The total amount of these additives generally ranges up to about 8.0 weight percent, preferably up to about 3.0 weight percent.

Antihistamines which may be used in compositions according to the invention include, without limitation, chlorpheniramine, triprolidine, diphenhydramine, doxylamine, pyrilamine, phenindamine, promethazine, cyproheptadine, azatadine, clemastine, carbinoxamine, tripelennamine, terfenadine, dexchlorpheniramine, brompheniramine, chlorcyclizine, diphenylpyraline, pheniramine and phenyltoloxamine, their pharmaceutically acceptable salts, and mixtures thereof in an amount between about 1.0 and about 4.0 percent by weight of the composition. In a preferred embodiment, hydrochloride salt of diphenhydramine is included in an amount of about 2.0 percent by weight of the composition.

Local anesthetics and/or antipruritics may be included in compositions according to the invention in an amount between about 0.1 percent by weight up to about 3.0 percent by weight. Exemplary anesthetics and antipruritics include, without limitation, dibucaine hydrochloride, procaine hydrochloride, hexothiocaine hydrochloride, benzyl alcohol, ethyl aminobenzoate, benzocaine, tetracaine hydrochloride, lidocaine, lidocaine hydrochloride, mepivacaine hydrochloride, cocaine hydrochloride, guatacaine hydrochloride, butanicaine hydrochloride, oxibutanicaine hydrochloride, meprylbutanicaine hydrochloride, pramoxine hydrochloride, piperocaine hydrochloride, chlorobutanol, meprylcaine hydrochloride, and mixtures thereof. In a preferred embodiment, the composition includes about 0.5 percent by weight dibucaine hydrochloride.

Antimicrobials, antibacterials and/or antiseptics may be included in the composition in an amount preferably up to about 1.0 percent by weight. Exemplary antimicrobials, antibacterials and antiseptics include, without limitation, benzalkonium chloride, benzethonium chloride, cetylpyridinium chloride, chlorhexidine, chlorhexidine gluconate, palmityl trimethyl ammonium chloride, thymol (including its isomers such as isopropyl methyl phenol), decanium chloride, thimerosal, mercurochrome, silverprotein, chloramine, sodium hypochlorite, potassium chlorite, iodine, sodium iodide, iodine tincture, povidone iodine, iodoform, oxidol, potassium permanganate, sodium perborate, ethanol, isopropanol, phenol, cresol, bithionol, acrinol, methylrosaniline chloride, nitrofurazone, resorcinol, domifen bromide, TEGO-51, chlorobutanol, salicylic acid, hexachlorophene, benzyl alcohol, benzoic acid, creosote, acriflavine, phenyl salicylate, sodium N-lauroyl sarcosinate, berberine chloride, berberine sulfate and mixtures thereof. In a preferred embodiment, benzethonium chloride and isopropyl methyl phenol are included, both in an amount of about 0.1 percent by weight of the composition.

Emulsifiers that may be included in the composition include any emulsifier approved for cosmetic use, including polyethylene glycol 20 sorbitan monolaurate (Polysorbate 20), polyethylene glycol 20 stearyl ether (Brij 78, Steareth 20), polyethylene glycol ether of lauryl alcohol (Laureth 23), polysorbate 80 (Tween 80), lecithin, etc. An emulsifier is generally present in an about between 0.1 and 2.0 percent by weight of the composition. In a particularly preferred embodiment, the emulsifier is Polysorbate 20, present in an amount of about 1.0 percent by weight of the composition. A mixture of two or more emulsifiers may also be used.

In some embodiments, a hydrolytically stable dimethicone copolyol is used in an amount of 0.1 percent by weight to about 4.0 percent by weight, as a detackifier to enhance the light, non-greasy feel of the composition. Dimethicone copolyol, available from the Dow Corning Company under product number DC 190, is a silicone glycol copolymer, soluble in water, alcohol and hydroalcoholic systems. Substantially equivalent detackifiers could also be used.

Preservatives in the composition may be selected from among those known in the art and commercially available for skin care product use. Such preservatives include Germaben II, commercially available from Sutton Laboratories, Chatham, N.J.

In a preferred embodiment, the invention is characterized as an aqueous skin-care composition consisting essentially of: about 70.0 to about 98.0 percent by weight water; about 0.1 to about 4.0 percent by weight water soluble hydroxyalkylcellulose polymer; about 1.0 to about 20.0 percent by weight glycerin; about 0. 1 to about 2.0 percent by weight of one or more vulnerary agents; about 0.1 to about 4.0 percent by weight of one more antihistamines; about 0.1 to about 1.0 percent by weight of one or more emulsifiers; about 0.01 to about 1.0 percent by weight of one or more antimicrobials, antibacterials or antiseptics; and about 0.01 to about 6.0 percent by weight of one or more antipruritics or anesthetics.

In another preferred embodiment, the invention is characterized as an aqueous skin-care composition comprising: about 80.0 to about 90.0 percent by weight water; about 1.0 to about 3.0 percent by weight water soluble hydroxyalkylcellulose polymer; about 0.1 to about 3.0 percent by weight pramoxine hydrochloride, about 0.01 to about 0.5 percent by weight benzethonium chloride, about 8.0 to about 15.0 percent by weight glycerin; about 0.01 to about 0.5 percent by weight aloe powder, about 0.1 to about 4.0 percent by weight dimethicone copolyol detackifier, and about 0.01 to about 2.0 percent by weight preservative.

Two parameters, in particular, are frequently used to assess the effectiveness of skin moisturizer compositions: moisture content of the skin and transepidermal moisture loss (TEWL). It is possible for a composition to effect an improvement in one of these parameters without improving the other. A surprising benefit observed in connection with compositions according to the invention has been that a substantial barrier to water loss is obtained without use of conventional occlusive water barrier ingredients while maintaining or improving moisture content of the skin.

Moisture content can be conveniently measured by measuring the electrical conductance of the skin, which indirectly provides a measurement of the skin's moisture content, as described for example in J. L. Leveque, et al., “Impedance Methods for Studying Skin Moisturization,” J. Soc. Cosmet. Chem., 34:419-428 (1983), herein incorporated by reference. The conductance measurements described herein were obtained using a SKICON®-2000 conductance meter, available from I. B. S. Co., Ltd. of Shizuokaken, Japan. The operation of this device is well known to those of ordinary skill in the art. Conductance is measured in units of millimhos.

TEWL measurements described herein were obtained using a DERMALAB® TEWL system, available from Cortex Technology of Handsund, Denmark. The instrument measures the temperature and relative humidity at two fixed points along an axis normal to the skin surface. A vapor pressure gradient is estimated and evaporative water loss determined therefrom. The operation of this unit is likewise known to those of ordinary skill in the art, as described for example in G. L. Grove, et al., “Comparative Metrology of the Evaporimeter and the DermaLab® TEWL Probe,” Skin Res. & Tech., 5:1-8 (1999), and G. L. Grove, et al., “Computerized Evaporimetry Using the Dermalab® TEWL Probe,” Skin Res. & Tech., 5:9-13 (1999), herein incorporated by reference. The units of evaporative water loss are expressed in g/m²/hr.

The following examples illustrate certain preferred embodiments and do not limit the scope of the invention defined by the claims.

### EXAMPLE

An exemplary formulation according to the invention was prepared from the following components:

TABLE 1

| | Weight |
|---|---|
| Ingredients | Percent |
| Deionized Water | 84.10 |
| Natrosol 250 HNF (Hydroxyethylcellulose) | 2.00 |
| Dibucaine HC1 | 0.50 |
| Diphenhydramine HCl | 2.00 |
| Benzethonium Chloride | 0.10 |
| Glycerin 96% | 10.00 |
| Tween 20 (Polysorbate 20) | 1.00 |
| Isopropyl Methyl Phenol | 0.10 |
| Allantoin | 0.20 |
| Total | 100.00 |

The hydroxyethylcellulose was added to the deionized water and heated to 60° C. The mixture was agitated until a homogeneous solution was obtained. Dibucaine, diphenhydramine, benzethonium chloride, glycerine, Polysorbate 20 and isopropyl methyl phenol were added, individually with sufficient mixing until a clear solution was obtained before the next component was added. Finally, allantoin was added and the composition was cooled to room temperature, with mixing.

Another particularly preferred embodiment, formulated in substantially the same way, has the formula set forth in Table 1a:

TABLE 1a

| | Weight |
|---|---|
| Ingredients | Percent |
| Deionized Water | 83.79 |
| Natrosol 250 HNF (Hydroxyethylcellulose) | 2.00 |
| Pramoxine HCl | 1.00 |
| Benzethonium Chloride | 0.20 |
| Glycerin (96%) | 10.00 |
| Germaben II (preservative) | 1.00 |
| Dimethicone Copolyol | 2.00 |
| Aloe Powder | 0.01 |
| Total | 100.00 |

To obtain objective indicia of the moisturization and water barrier properties of the formulation in Table 1, human subjects prepared by washing their forearms three times daily with soap. Subjects showing a 3-unit increase in transepidermal water loss (TEWL) over a one-week period were selected for the study. A baseline measurement was taken of the subjects. Then 2 mg/cm² of the above composition was applied to the volar forearm of each subject. Moisture content and TEWL measurements were taken at the start and at 8 hours using the above-described devices.

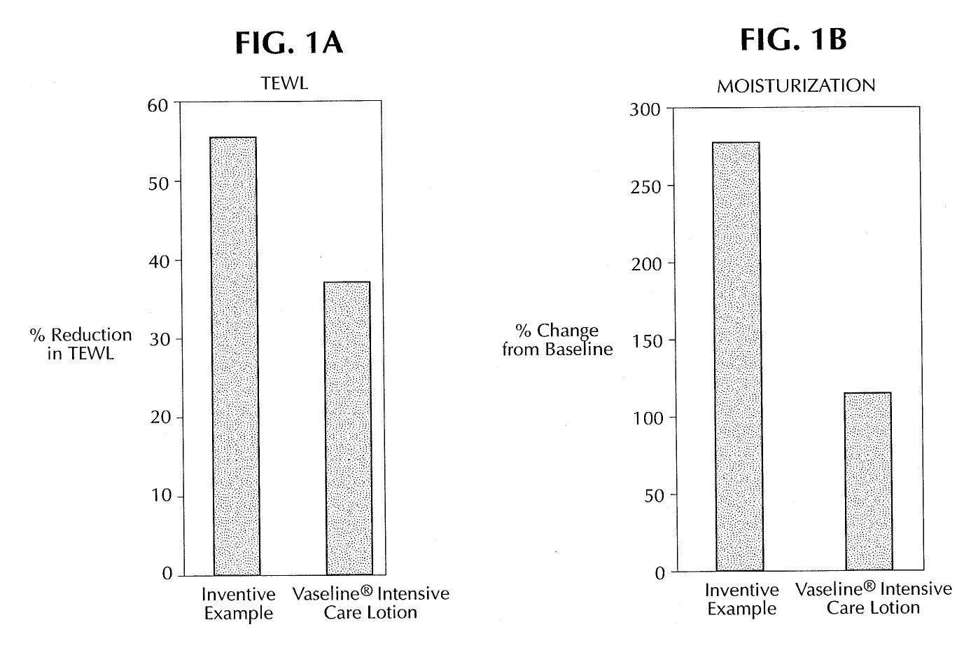

FIG. 1 shows the improvement over baseline of the moisturizing properties and transepidermal water loss properties of the inventive composition compared to those of Vaseline® Intensive Care Lotion, a conventional formulation containing occlusive hydrophobic components. The primary occlusive active ingredient of Vaseline® Intensive Care Lotion is petrolatum. Unexpectedly, the inventive moisturizing composition exhibits a substantial ability to reduce transepidermal water loss, notwithstanding the absence of occlusive components.

### COMPARATIVE EXAMPLES

Although glycerin has been used in skin moisturizing compositions (including, for example, Vaseline® Intensive Care Lotion), it is used as a humectant, to increase the moisture content of the skin. The inventors herein have performed tests to determine whether glycerin also has a water barrier effect in the inventive composition.

A composition substantially identical to the composition of Table 1 was prepared, except that no glycerine was used (the balance being made up with deionized water). As in the foregoing comparison 2 mg/cm² of the glycerine-free formulation and a like dosage of the formulation shown in Table 1 were applied to the forearms of the eight subjects.

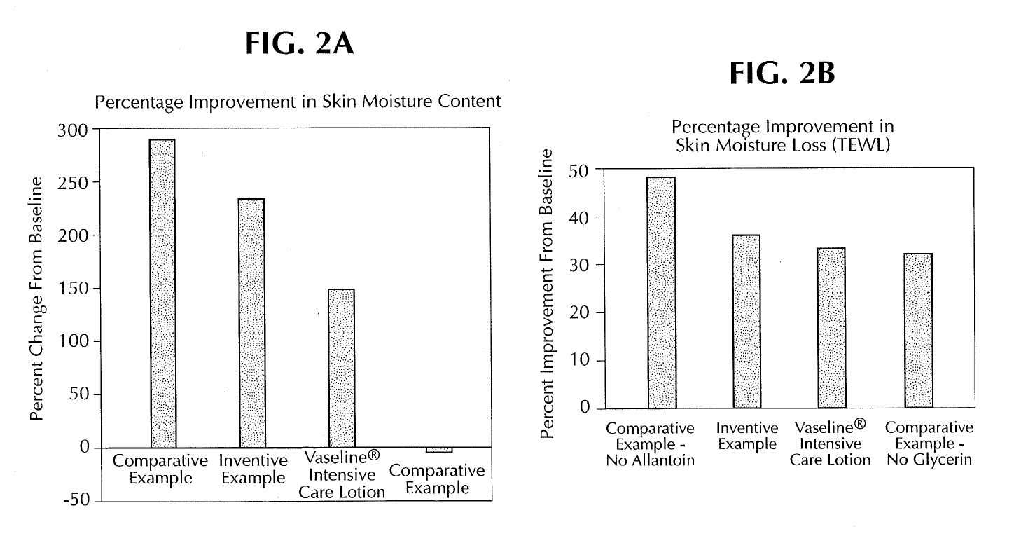

As shown in FIG. 2, the moisture content of the skin dropped precipitously in those subjects who were administered the glycerin-free composition. By contrast, the water barrier properties of the respective compositions were substantially unaffected. Thus, the inventors have concluded that glycerine does not possess substantial water barrier properties.

The vulnerary agent, allantoin, likewise, has been described in the art as increasing water-binding properties. On this basis one might expect that it would exhibit water-barrier properties as well. To the contrary, the inventors herein have found that a composition prepared according to Table 1 above, excluding allantoin (balance deionized water), surprisingly exhibited increased moisturization and water barrier properties as shown in FIG. 2.

Without wishing to be bound by any particular theory, the inventors herein believe that a water soluble hydroxyalkylcellulose polymer of the invention, such as hydroxyethylcellulose, heretofore used in skin care compositions only as a gelling agent, exhibits significant water-barrier properties. It is not believed that skin moisturizers were formulated previously having hydroxyethylcellulose or its equivalent as the primary water-barrier agent.

Two commercially available gelling agents were tested as individual ingredients for their moisturization and water-barrier properties, a carbomer available from Noveon, Inc., and NATROSOL 250 HNF®, a water soluble hydroxyethylcellulose according to the invention. As in the foregoing examples, eight subjects were selected based upon a three-unit increase in TEWL over a one-week period.

Upon selection of the eight subjects, 0.05 cc of each gelling agent was applied to the subjects' skin at different times, and the moisture content of skin measured at the start and after eight hours. As shown in Table 2, no significant change was observed in terms of percent change from baseline in the skin moisture content in the subjects evaluated.

TABLE 2
PERCENT CHANGE FROM BASELINE
Moisture Content

| Carbomer | Hydroxyethylcellulose |
|---|---|
| 14.0 | 38.5 |
| −10.4 | −10.2 |
| −23.8 | −28.3 |
| 11.4 | 21.7 |
| 47.4 | −9.9 |
| −22.6 | −2.8 |
| 28.7 | 40.7 |
| −34.2 | −12.2 |

Surprisingly, however, when TEWL measurements were made on the same subjects, the hydroxyethylcellulose was found to create an unexpectedly significantly greater barrier to moisture, as shown in Table 3 below.

TABLE 3
PERCENT CHANGE FROM BASELINE
(TEWL)

| Carbomer | Hydroxyethylcellulose |
|---|---|
| 22.6 | −6.6 |
| 8.5 | −5.6 |
| −1.2 | −40.3 |
| −9.1 | −10.1 |
| 6.6 | −21.4 |
| −3.8 | −21.4 |
| −9.3 | −29.7 |
| 7.8 | −32.3 |

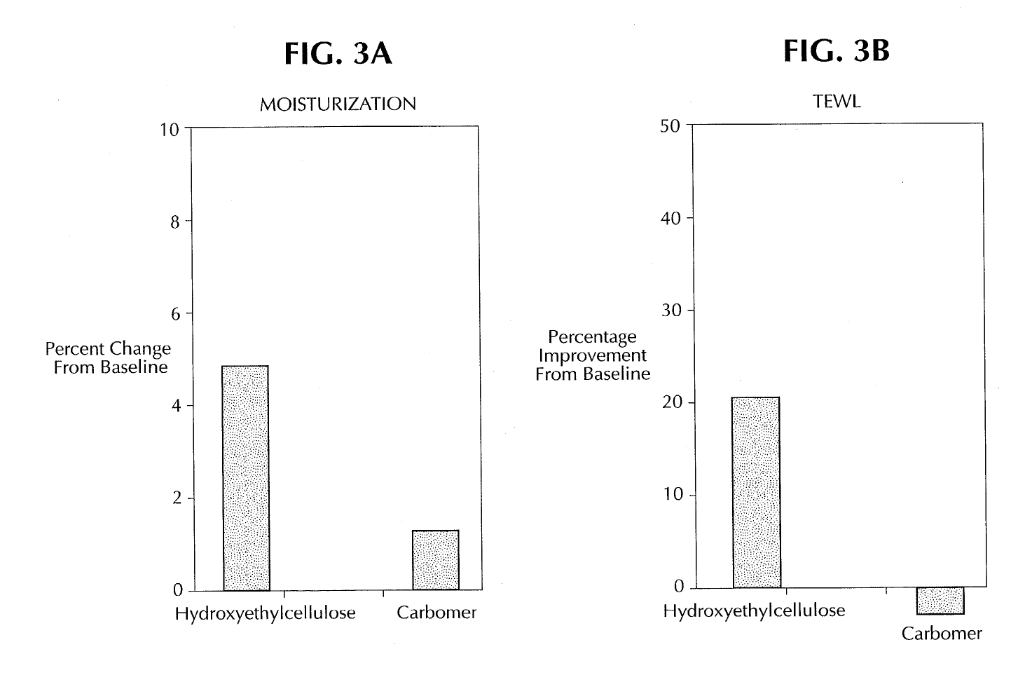

These results are depicted graphically in FIG. 3.

An aspect of the invention is that a water-based skin moisturizer composition having substantial water-barrier properties is provided in which a water soluble hydroxyalkylcellulose polymer, such as hydroxyethylcellulose, is the primary water-barrier ingredient.

Other modifications and embodiments will be apparent to those of ordinary skill in this art. This invention is not to be limited except as set forth in the following claims.

What is claimed is:

1. An aqueous skin moisturizing composition having substantial water-barrier properties comprising a water soluble hydroxyalkylcellulose polymer, a skin moisturizer and water, and being free of a film-forming polyglyceryl methacrylate polymer, occlusive fats or oils.

2. The composition of claim 1, wherein said composition is in the form of a gel.

3. The composition of claim 1, wherein said water soluble hydroxyalkylcellulose polymer is hydroxyethylcellulose.

4. The composition of claim 1, wherein said skin moisturizer is an alkylenepolyol.

5. The composition of claim 4 wherein said alkylenepolyol is glycerin.

6. The composition of claim 1, further comprising an agent selected from the group consisting of vulnerary agents, antihistamines, emulsifiers, antipruritics, antimicrobial, antibacterial or antiseptic agents, stabilizers, preservatives, and mixtures thereof.

7. The composition of claim 6, comprising allantoin in an amount less than about 1.0 percent by weight of the composition.

8. The composition of claim 6, comprising diphenhydramine hydrochloride in an amount less than about 1.0 percent by weight of the composition.

9. The composition of claim 6, comprising dibucaine, or a cosmetically acceptable salt thereof, in an amount less than about 1.0 percent by weight of the composition.

10. The composition of claim 6, comprising isopropyl methyl phenol in an amount less than about 1.0 percent by weight of the composition.

11. An aqueous skin moisturizing composition according to claim 1, comprising: about 80.0 to about 90.0 weight percent deionized water, about 1.0 to about 3.0 weight percent hydroxyethylcellulose, about 0.1 to about 3.0 weight percent pramoxine hydrochloride, about 0.01 to about 0.5 weight percent benzethonium chloride, about 8.0 to about 15.0 weight percent glycerin, about 0.01 to about 0.5 weight percent aloe powder, about 0.1 to about 4.0 weight percent of a dimethicone copolyol detackifier, and about 0.01 to about 2.0 weight percent preservative.

12. An aqueous skin moisturizing composition according to claim 1, comprising: about 80.0 to about 90.0 weight percent deionized water, about 1.0 to about 3.0 weight percent hydroxyethylcellulose, about 8.0 to about 15.0 weight percent glycerin, about 0.1 to about 1.0 weight percent dibucaine hydrochloride, about 1.0 to about 3.0 weight percent diphenhydramine hydrochloride, about 0.01 to about 0.5 weight percent benzethonium chloride, about 0.1 to about 2.0 weight percent polysorbate 20, about 0.01 to about 0.5 weight percent isopropyl methyl phenol, and about 0.1 to about 0.5 weight percent allantoin.

13. An aqueous skin-care composition consisting essentially of:
about 70.0 to about 98.0 percent by weight water;
about 0.1 to about 4.0 percent by weight water soluble hydroxyalkylcellulose polymer;
about 1.0 to about 20.0 percent by weight glycerin;
about 0.1 to about 2.0 percent by weight of one or more vulnerary agents;
about 0.1 to about 4.0 percent by weight of one more antihistamines;
about 0.1 to about 1.0 percent by weight of one or more emulsifiers;
about 0.01 to about 1.0 percent by weight of one or more antimicrobials, antibacterials or antiseptics; and
about 0.1 to about 6.0 percent by weight of one or more antipruritics or anesthetics.

14. The composition of claim 13, including about 84.1 weight percent deionized water, about 2.0 weight percent hydroxyethylcellulose, about 10.0 weight percent of a 96 weight percent solution of glycerin, about 0.5 weight percent dibucaine hydrochloride, about 2.0 weight percent diphenhydramine hydrochloride, about 0.10 weight percent benzethonium chloride, about 1.0 percent polysorbate 20, about 0.10 weight percent isopropyl methyl phenol, and about 0.20 weight percent allantoin.

15. A method of reducing transepidermal water loss comprising the step of applying to the skin of a subject in need thereof an effective amount of an aqueous skin moisturizing composition comprising a water soluble hydroxyalkylcellulose polymer, a skin moisturizer and water, and free of film-forming polyglyceryl methacrylate polymer, occlusive fats or oils.

16. The method of claim 15, wherein said composition is a gel comprising about 1 to about 3 percent by weight hydroxyethylcellulose, about 80 to about 90 percent by weight deionized water, about 8 to about 12 percent by weight glycerin.

17. The method of claim 16, wherein said composition further comprises at least one additive selected from the group consisting of vulnerary agents, antipruritics, anesthetics, stabilizers, preservatives, antimicrobials, antibacterials, antiseptics, and emulsifiers.
